(19) United States
(12) Patent Application Publication
(10) Pub. No.: US 2010/0173884 A1
(43) Pub. Date: Jul. 8, 2010

(54) USE OF A PHYTOSTEROL CONTAINING COMPOSITION FOR THE TREATMENT OF LUTS

(75) Inventors: Lars Kärnerud, Tenhult (SE); Arata Paul Yamasaki, Tokyo (JP); Claus Tufvesson, Vejbystrand (SE)

Correspondence Address: MERCHANT & GOULD PC, P.O. BOX 2903, MINNEAPOLIS, MN, 55402-0903, US

(73) Assignee: Natumin Pharma AB, Huskvarna (SE)

(21) Appl. No.: 12/518,851

(22) PCT Filed: Dec. 6, 2007
(86) PCT No.: PCT/SE2007/050948

§ 371 Date: Mar. 10, 2010

Related U.S. Application Data
(60) Provisional application No. 60/874,119, filed on Dec. 11, 2006.

(30) Foreign Application Priority Data
Dec. 11, 2006 (SE) 0602654-6

(51) Int. Cl. A61K 31/575; A61P 13/10

(52) U.S. Cl. 514/182

(57) ABSTRACT

The invention relates to the use of a composition comprising an oil-extract and a water-soluble extract, wherein said extracts are obtainable from plant materials and said extracts comprise phytosterols, for the manufacturing of a medicament for the treatment of females suffering from LUTS as a consequence of Interstitial Cystitis and/or stress or urge incontinence.

### FIELD OF INVENTION

The invention relates to the use of a composition comprising an oil-extract, wherein said oil-extract is obtainable from a plant material and said oil-extract comprises phytosterols for the manufacturing of a medicament for the treatment of females suffering from lower urinary tract symptoms, LUTS, as a consequence of Interstitial Cystitis and/or stress or urge incontinence.

### BACKGROUND OF INVENTION

The group of women suffering from LUTS is increasing and the treatments available are for example different medical treatments with different pharmaceutical compositions such as estrogen or anticholinergic spasmolytics, surgical treatment, or no treatment as the woman accepts the problem. Often the pharmaceutical compositions have side effects. The LUTS may be varying, e.g. frequency, urgency, nocturia, urinary leakage upon sneezing, and pelvic/perineal or bladder pain.

The LUTS may be a consequence of stress or urge incontinence or by Interstitial Cystitis (IC). The diagnostic criteria for IC are urgency, frequency, and bladder or pelvic/perineal pain in the absence of bacterial infection or other definable pathology. Pain at bladder filling and glomerulations in the bladder wall upon hydrodistention are also suggestive of IC. Due to the many diffuse symptoms it is sometimes difficult to diagnose if a woman is suffering from IC. Since there is such a large group suffering from LUTS there is an increasing demand for alternative medicaments, which the woman may obtain and try without being diagnosed by a doctor. LUTS are often considered improper to talk of and the women often feel ashamed and need to change life style or to use different absorbing products, such as sanitary pads. This may cause worries about leakage and smell.

U.S. Pat. No. 6,197,309 discloses a composition comprising antioxidants, immunoboosters, phytosterols, etc., etc., that is beneficial to bladder health in women. The effect is said to be obtained by the presence of the antioxidants and immunity boosters.

Other compositions have been disclosed in the art, which compositions comprise an oil-extract and a water-soluble extract, for treatment of prostatitis and benign prostate.

### SUMMARY OF THE INVENTION

The invention relates to a medicament, which provides a new improved mode of treatment for women, suffering from lower urinary tract symptoms (LUTS). By the use of the new invented medicament the females do not need to be worried about leakage or smell from urine or where the closest bathroom is located. In a first aspect the invention relates to the use of a composition comprising an oil-extract, wherein said oil-extract is obtainable from a plant material and said oil-extract comprises phytosterols, for the manufacturing of a medicament for the treatment of women suffering from LUTS as a consequence of Interstitial Cystitis and/or stress or urge incontinence.

By providing such a new medicament women suffering from LUTS may be cured or the symptoms may at least be reduced. The quality of life of the women will thereby be much improved and the consumption of sanitary products decreased.

Further advantages and objects with the present invention will be described in more detail below.

### DETAILED DESCRIPTION OF THE INVENTION

### Definitions

In the context of the present application and invention, the following definitions apply:

The term “oil-extract” is intended to mean an oil part or fraction obtainable from a plant material such as from one or more plants species from the families Graminae and/or Pinacea. For example, the oil-extract may be obtained from pollen. Said oil-extract includes one or more phytosterols, such as those mentioned below.

The term “water-soluble extract” is intended to mean a water-soluble extract obtainable from one or more plants species, such as obtained from the families Graminae and/or Pinacea. Said water-soluble extract may be obtained from pollen. Said water-soluble extract includes one or more of superoxide dismutase (SOD) mimics, flavonoids, tannins or polyphenols.

It has surprisingly been found that the use of a composition comprising an oil-extract, wherein said oil-extract is obtainable from a plant material and said oil-extract comprises phytosterols for the manufacturing of a medicament for the treatment of women suffering from LUTS. The amounts of the phytosterols are different between different plant species and therefore different amounts needs to be used if the oil-extract is obtained from different sources. For example the oil extract used in the examples contains about 5-15%, such as 6, 7, 8, 9, 10, 11, 12, 13 or 14% or 8-12% (weight/weight) of phytosterols. The phytosterols may for example be selected from the group consisting of fucosterol, beta-sitosterol, campesterol, stigmasterol or any other source, which contains phytosterols. The phytosterols may be synthetic as well as semi-synthetic. The oil-extract may be obtained from plants from the Graminae and/or Pinacea families, such as being obtained from pollen from said species.

Additionally the composition may comprise a water-soluble extract, wherein said water-soluble extract is obtainable a plant material such as from plants from the Graminae and/or Pinacea families, such as pollen from said species. By using a mixture between an oil-extract and a water-soluble extract an improved composition is obtained which gives a better effect upon use, i.e., to treat LUTS in women suffering from conditions, such as Interstitial Cystitis and/or stress or urge incontinence. The technical effect obtained by using a mixture between an oil-extract and a water-soluble extract is that the accessibility of phytosterols is increased. Phytosterols are included in a large group of substances that normally are not included in a water-soluble form. It has however been shown (for example in Borel, P. “Factors affecting intestinal absorption of highly lipophilic food microconstituents (fat-soluble vitamins, carotenoids and phytosterols)”, Clin Chem Lab Med. 2003 August; 41(8):979-94) that the up take of phytosterols varies, depending on the sort of phytosterol and the composition of the matrix in which the phytosterol is present. Thus, the present inventors assumes that it is possible to control the up take of phytosterols by adapting the composition in which the phytosterols are present. Without being bound to theory, the present inventors believe that the normally hydrophobic phytosterols forms micells in the hydrophilic phase in a composition of hydrophobic and hydrophilic phases. In this way the up-take of phytosterols is believed to increase, since the phytosterols are present in both phases whereby the phytosterols can be absorbed by cells to a larger extent, resulting in the improved composition and the increased effect described above.

The oil-extract and/or the water-soluble extract may be obtained from at least one plant selected from the group consisting of timothy, saw palmetto, maize, rye, wheat, wild chervil, Pygeum (Prunus africanum, Pygeum africanum) and pumpkin (Cururbita Pepo), such as pumpkin seed or being a mixture thereof.

In one embodiment the composition comprises silicon dioxide. Silicon dioxide being present, to bind and protect the oil-extract from being oxidised.

Examples of oil-extracts are EA5/EA10 from Allergon AB, Sweden, GBX from Cernelle AB, Sweden and GFX, NAX from Graminex US.

Examples of water-soluble extracts are P2, PI82 and GCFEM from Allergon AB, Sweden.

Said oil-extract may be included in amounts to give a daily dosage of from about 1 to about 20 mg of phytosterols, such as to give a daily dosage of about 2, 3, 4, 5, 6, 7, 8, 9, 10, 11, 12, 13, 14, 15 or 16 mg. The dose may be between 4 to 20 mg such as between 8 to 16 mg or between 10-12 mg.

Said medicament may be in the form of a tablet, capsule or a powder. The ratio between the water-soluble extract may from about 1:4 to about 1:20, such as 1:10.

Accordingly the medicament may be administrated in a single does or in multiple doses and it may also be administrated as an initial higher dose or doses which after a period of time are reduced to a lower level of administration.

The oil extract and/or water-soluble extract may be produced using the method disclosed in U.S. Pat. No. 3,360,437 with the modification that no mold was used. The extracts may also be bought from one or more manufactures such as those mentioned above.

Following examples are intended to illustrate, but not to limit, the invention in any manner, shape, or form, either explicitly or implicitly.

### EXAMPLES

### Example 1

The oil-extract named EA5/EA10 and the water-soluble extract P2 were bought from Allergon AB, Välingevägen 309, 262 92 Ängelholm, Sweden.

| Ingredients | Amount | |
|---|---|---|
| Tablet A | | |
| Pollen extract, water-soluble | 140.00 | mg |
| Pollen extract, oil | 8.00 | mg |
| Microcrystalline cellulose | 177.00 | mg |
| Lactose | 170.00 | mg |
| Colloidal silicon dioxide | 3.00 | mg |
| Magnesium stearate | 2.00 | mg |
| Total: | 500.00 | mg |
| Dosage | 1-2 | capsules per day |
| Capsule B | | |
| Pollen extract, water-soluble | 70.00 | mg |
| Pollen extract, oil | 8.00 | mg |
| Isomalt | 200.00 | mg |
| Microcrystalline cellulose | 117.00 | mg |
| Colloidal silicon dioxide | 3.00 | mg |
| Magnesium stearate | 2.00 | mg |
| Total: | 400.00 | mg |
| Dosage | 1-2 | tablets per day |
| Soft capsule C | | |
| Pollen extract, water-soluble | 70.00 | mg |
| Pollen extract, oil | 16.00 | mg |
| Vegetable oil | 200 | mg |
| Total: | 286.00 | mg |
| Dosage | 1 | capsule per day |

### Example 2

A trial was performed to investigate the efficacy of the invented composition.

A trial was performed on 41 women aged 30 to over 60 years old all suffering from LUTS, wherein 40 women finalised the trial.

9 women were between 30-39 years old. 10 were 40 to 49 years old, 14 were 50-59 years old and 8 were over 60 years old.

The women were given tablet A from example 1 every day and the women were evaluated on regular basis after 2 weeks, one month and 2 months. The symptoms that were evaluated were urine leakage by sneezing, urine leakage before reaching toilet, frequently go to toilet and feel bladder is not completely empty. The different symptoms were scored as % values on how many women that ended up in each group.

The results are shown below.

| | | Significantly | Slightly | No |
|---|---|---|---|---|
| | Cured | improved | improved | improvement |
| Symptoms after 2 | | | | |
| weeks of treatment | | | | |
| Urine leakage by | 7.7 | 15.4 | 38.5 | 38.5 |
| sneezing | | | | |
| Urine leakage | 9.1 | 13.6 | 36.4 | 40.9 |
| before reaching | | | | |
| toilet | | | | |
| Frequently go to | 4.3 | 4.3 | 39.1 | 52.2 |
| toilet | | | | |
| Felt bladder is not | 9.1 | 9.1 | 36.4 | 45.4 |
| completely empty | | | | |
| Symptoms after 1 | | | | |
| month of treatment | | | | |
| Urine leakage by | 7.7 | 25.6 | 41 | 25.6 |
| sneezing | | | | |
| Urine leakage | 33.3 | 33.3 | 16.7 | 16.7 |
| before reaching | | | | |
| toilet | | | | |
| Frequently go to | 4.3 | 34.8 | 34.8 | 26.1 |
| toilet | | | | |
| Felt bladder is not | 0 | 45.5 | 27.3 | 27.3 |
| completely empty | | | | |
| Symptoms after 2 | | | | |
| months of | | | | |
| treatment | | | | |
| Urine leakage by | 17.9 | 35.9 | 38.5 | 7.7 |
| sneezing | | | | |
| Urine leakage | 50 | 33.3 | 16.7 | 0 |
| before reaching | | | | |
| toilet | | | | |
| Frequently go to | 4.3 | 52.2 | 21.7 | 21.7 |
| toilet | | | | |
| Felt bladder is not | 0 | 45.5 | 36.4 | 18.2 |
| completely empty | | | | |

The results from the trial above shows that during the treatment with the tablet 1 from example 1 there is an increase in the women that are cured or have less of the lower urinary tract symptoms, which are typical symptoms of IC and stress or urge incontinence. The women suffering from urine leakage by sneezing was 38.5% after 2 weeks and decreased to 7.7% after 2 months and 17.9% were cured after 2 months. The women suffering from urine leakage before reaching toilet was 40.9% after 2 weeks treatment and all were cured after 2 months. The women suffering from that they frequently needed to visit the toilet was 52.5% after 2 weeks of treatment and solely 21.7% after 2 months and the women suffering from that they felt that the bladder was not completely empty decreased from 45.5% after 2 weeks of treatment to 18.2% after 2 months. I.e., the treatment was a success and all symptoms were decreased. The treatment outcome also suggested that a prolonged treatment probably would result in an even better result.

1. Use of a composition comprising an oil-extract and a watersoluble extract, wherein said extracts are obtainable from plant materials and said extracts comprise phytosterols for the manufacturing of a medicament for the treatment of females suffering from lower urinary tract symptoms (LUTS).

2. Use according to claim 1, wherein said phytosterols are selected from the group consisting of fucosterol, beta-sitosterol, campesterol and stigmasterol.

3. Use according to claim 1, wherein said oil-extract is obtainable from the Graminae and/or Pinacea families.

4. Use according to claim 3, wherein said oil-extract is obtainable from pollen.

5. Use according to claim 1, wherein said water-soluble extract is obtainable from the Graminae and/or Pinacea families.

6. Use according to claim 5, wherein said water-soluble extract is obtainable from pollen.

7. Use according to claim 1, wherein said oil-extract and/or water-soluble extract is/are obtained from at least one plant selected from the group consisting of timothy, saw palmetto, maize, rye, wheat, wild chervil, pygeum africanum and pumpkin seed.

8. Use according to claim 1, wherein said composition comprises silicon dioxide.

9. Use according to claim 1, wherein said oil-extract is included in the medicament in amounts such as to give a daily dosage of from about 1 to about 20 mg of phytosterols.

10. Use according to claim 1, wherein said oil-extract is included in the medicament in amounts such as to give a daily dosage of from about 4 to about 20 mg of phytosterols.

11. Use according to claim 8, wherein said oil-extract is included in the medicament in amounts to give a daily dosage of from about 8 to about 16 mg of phytosterols.

12. Use according to claim 1, wherein said medicament is in the form of a tablet, capsule or a powder.

13. Use according to claim 1, wherein the ratio between the oil-extract and the water-soluble extract is from about 1:4 to about 1:20.

14. Use according to claim 1, wherein the daily dosage of the medicament is administrated in a single dose or in multiple doses.

15. Use according to claim 1, wherein said LUTS are a consequence of Interstitial Cystitis and/or stress or urge incontinence.
